(19) United States
(12) Patent Application Publication
(10) Pub. No.: US 2007/0077226 A1
(43) Pub. Date: Apr. 5, 2007

(54) Gutless adenovirus vector and the construction method thereof

(75) Inventors: Xinyuan Liu, Shanghai (CN); Zifel Pei, Shanghai (CN); Binghua Li, Shanghai (CN); Jinfa Gu, Shanghai (CN); Weiguo Zou, Shanghai (CN); Lanying Sun, Shaghai (CN)

Correspondence Address: PERKINS COIE LLP, POST OFFICE BOX 1208, SEATTLE, WA, 98111-1208, US

(73) Assignee: SHANGHAI INSTITUTES FOR BIOLOGICAL SCIENCES CHINESE ACADEMY OF SCIENCES, Shanghai (CN)

(21) Appl. No.: 10/559,008

(22) PCT Filed: May 28, 2004
(86) PCT No.: PCT/CN04/00556

§ 371 Date: May 12, 2006

(30) Foreign Application Priority Data
May 30, 2003 (CN) 03128996.7

(51) Int. Cl. A61K 48/00; C12N 15/861

(52) U.S. Cl. 424/93.2; 435/456; 977/802

(57) ABSTRACT

The present invention disclosed a kind of gutless adenovirus vector and the construction method thereof. Two structural independent but functional related cassettes, the trans-activator (TA) and anti-tumor cassette, are both carried by the gutless vector. hTERT promoter restricts the expression of TA only in tumor cells, and RU486, associated with TA, regulates the expression of interesting gene: when needed, add the RU486 and the gene expression is on, and when not needed, remove the RU486 and the gene expression is off. Tumor-specificity and small molecule regulation of the vector spare the toxicity to the normal tissue caused by the foreign gene product and endow the gene's long lifetime expression in vivo. The vector of the present invention shows many advantages over traditional adenovirus vectors in targeting, gene regulation and expression lifetime.

### FIELD OF THE INVENTION

The present invention belongs to the field of biotechnology, and in particular, relates to novel gutless adenovirus (GL-Ad) vectors that show tumor-specific, small molecule regulated and long lifetime foreign gene expression, and the construction method thereof.

### BACKGROUND OF THE INVENTION

The gene therapy is a high bio-technique to deliver therapeutic genes into patients which is emerging in the near 10 years. More than 60% of all gene therapy protocols are for cancer gene therapy. Gene therapy is thought to be a hope that mankind finally conquer the tumor.

Vectors for gene therapy are divided into two types: viral vectors and non-viral vectors. Viral vectors include adenovirus, adeno-associated virus (AAV), retrovirus, lentivirus and herpes virus. Non-viral vectors include naked DNA or capsulated DNA with liposome or other materials. Gene therapy by viral vectors is developed rapidly in recent years. Therefore, adenoviral vectors for tumor gene therapy are becoming most popular because these vectors can infect many kinds of cells including dividing and non-dividing cells, and purified vectors with high titers can be obtained by large culture.

The wild-type adenoviruses (Ads) are DNA viruses with a double-stranded linear genome of 36 kb. The genome is divided into early functional transcription regions (E1A, E1B, E2A, E2B, E3, and E4 genes), which encode regulating proteins, and late functional transcription regions (L1 to L5 regions), which encode the viral structural proteins. Knowledges about adenovirus type 2 and type 5 are most. Adenoviruses used as vector systems are usually constructed from adenovirus type 2 and type 5. Adenovirus vectors (Ad vectors) have a cloning capacity of about 2 kb foreign DNA (up to 105% of the wildtype genome). Deletions in the nonessential regions can further increase the cloning capacity. But smaller viral genomes less than 75% of the viral genome (27 kb) tend to undergo rearrangement and a stuffer sequence must be inserted.

The first generation Ad vectors are constructed by deletion in adenovirus genome E1 region (sometimes also including deletion in E1 and E3 at the same time). The E1 region is located adenovirus genome at 1.0-10.6 mu place, for viral duplication essential region. Adenovirus with E1 region deletion only can duplicate in the E1 gene product representation cells (for example 293 cells or other similar cells), therefore such vectors are also called the unduplicated vectors. The maximum possible size of deletions in the E1 region may reach 3.2 kb. The E3 region also may be deleted and the deleted length be possible to reach 3.1 1kb. The E3 region has been shown to be nonessential for viral replication. So Ad vectors with E3 region deletion still have the duplication ability.

At present the most popular used first generation Ad vectors are with the deletions in E1 and E3 regions at the same time, which have the capacity for the insertion of foreign genes up to approximately 8.3 kb. The foregin genes inserted in first generation Ad vectors show very high efficiency in transfer and expression into a large variety of cell types. But at the same time the first generation Ad vectors also express the viral proteins itself at certain level, so it is easy to cause the host cell immune response to the viral proteins and the virus is eliminated, and the foregin genes expression will be restricted. When infection is performed once more with these vectors, these vectors will be eliminated very quickly by already formed immune reply and expression of the foregin genes may be failed. In order to lengthen the foregin genes expression time carrying by Ad vectors, some novel Ad vectors must be developed.

Second generation Ad vectors: The immunogenicity of Ad mainly produced from the expression of viral later period proteins and expression of the later period proteins is regulated by early gene products in great degree. Therefore the second generation Ad vectors are constructed on the foundation of first generation Ad vectors for further deletions of E2 and E4 genes to weaken the early gene functions and reduce the later period proteins expression. The second generation Ad vectors have deletions in the E1 and E3 regions accompanied by deletions in the E2 or E4 regions, therefore the inserted foreign genes may be as large as 14 kb. The second generation Ad vectors although reduced own immunogenicity in certain degree, but its duplication ability decreased very much compared to the first generation Ad vectors. At the same time, its immunogenicity is unable to eliminate completely. Moreover because the preparation methods of second generation Ad vectors are different, the second generation Ad vectors might not as such favored for scientists as first generation Ad vectors, therefore it is little used.

The third generation Ad vectors are called the gutless Ad vectors (GL-Ad), which have the deletions of all adenovirus genome's code regions, only retain its reverse terminal repetition sequence (ITR) and the viral packing signal (Ψ). Because of complete deletions of Ad genome's code regions, the virus lost the packing ability. In order to achieve its effective packing length, some irrelevant DNA sequence (for example intron) must be inserted. After the foreign genes are inserted, the total length of recombinant vector's genome can not to be smaller than 30 kb, but cannot surpass 38 kb. Because GL-Ad eliminated its immunogenicity completely and enable long-term expression of inserted foreign genes, these characteristics enabled GL-Ad to become one of best vectors for gene therapy. But GL-Ad itself cannot duplicate because of complete deletions of Ad genome's code regions. The generation of virus granules has to produce in the special packing cell, and must coinfected with the helper virus. At present packing cell line used in GL-Ad is 293Cre4, which the Cre recombinase gene is transfered into the ordinary 293 cell lines and enables this cell line stably express the recombinase. The Cre recombinase can distinguish the size of the 34 bp loxP sequence. If some gene or some DNA fragment has a loxP sequence at both ends respectively, this gene or the DNA fragment is cut down by the Cre recombinase for the homologous recombination of loxP sequence. A schematic overview of Cre recombinase's function on the helper virus is indicated in FIG. 2.

### PURPOSE OF THE INVENTION

One purpose of the invention is to provide novel tumor-specific, small molecule regulated and long lifetime foreign gene expressed GL-Ad vectors which overcome the disadvantages of traditional adenovirus vectors in poor tumor-targeting, no gene regulation and short foreign gene expression lifetime.

The second purpose of the invention is to provide the construction methods of the novel tumor-specific, small molecule regulated and long lifetime foreign gene expressed GL-Ad vectors.

### SUMMARY OF THE INVENTION

In order to achieve the above goal, the cancer-targeting and inducible gene-expression GL-Ad of the invention contains two expressing cassettes. The two cassettes are the trans-activator(TA) cassette (cassette-1) and the anti-cancer gene expression cassette (cassette-2) repectively. GL-Ad is used as the vector of the two expressing cassettes.

The cassette-1 includes: (1) tumor-specific promoter; (2) the trans-activator(TA) consists of a mutated human progesterone receptor ligand binding domain(LBD) fused to the yeast GAL4 DNA binding domain and transcriptional activation domain of the human NF-kB p65 protein, and the ending signal.

The cassette-2 includes: (1) the yeast GAL4 upstream 17mer×4 sequence; (2) TATA cassette; (3) anti-cancer gene downstreasm the TATA cassette; (4) and the ending signal.

According to the present invention, methods for construction of the novel GL-Ad with cancer-targeting and adjustable expression of genes comprising the following steps:

1. Construction of the Plasmid pRS-hTERT/Trail

1-1. Construction of the Trans-Activator(TA) Cassette (Cassette-1)

The hTERT was cut from pShuttle/hTERT and cloned into the unique KpnI site of pRS-17 to generate pRS-hTERT, orientatining the hTERT to regulate expression of TA.

1-2. Construction of the Anti-Cancer Gene Expression Cassette (Cassette-2)

TRAIL was cut from pCA13-Trail with HindIII/XbaI and cloned into a shuttle plasmid pGL3-Basic at HindIII/XbaI site to form plasmid pGL3-TRAIL. pGL3-TRAIL was cut with NheI/ClaI and digested fragment was inserted into the corresponding site of pRS-hTERT to make pRS-hTERT/Trail.

2. Construction of the Packaging Plasmid pGL-hTERT/Trail

The two cassettes were cut out with NotI from plasmid pRS-hTERT/Trail and cloned into the unique EagI site of pGL to make the final packaging plasmid pGL-hTERT/Trail.

3. Package of Gutless Adenoviral Vector

Linearized pGL-hTERT/Trail was cotransfected/infected the 293Cre4 cell with the helper virus H14 (Microbix Biosystem Inc., Toronto) to package the novel GL-Ad with cancer-targeting and adjustable expression of genes.

4. Large Scale Preparation and Purification of GL-Ad

After several rounds of plaque purification and the cloning is correct, this GL-Ad stock is used for large scale preparation. 293Cre4 cells are infected with this GL-Ad stock. When cells show complete CPE, cells are harvested. Purified GL-Ad vector is obtained from CsCl gradient centrifugation and freeze in aliquots at −80° C.

In this method of gene-viral vector construction, GL-Ad vectors with no immunogenicity are used. This characteristic makes the GL-Ad vectors escape from immunity system attack, then enables the extraneous genes which carries effectively express in the tumor cells for a long time.

These noval GL-Ad vectors of this invention, with cancer-targeting and adjustable anti-tumor genes expression, has the following beneficial effect:

1. This invention provides one kind of GL-Ad vectors, that the goal genes inserted in can express specificially in the tumor cells but not in the normal cells in vitro the cell experiment;

2. This invention provides one kind of GL-Ad vectors, that the promoter to regulate TA can be replaced. According to the characteristic of special promoter, the expression of goal genes may be in the most types tumor cells or may only be restricted in certain specific tumor cells;

3. This invention provides one kind of GL-Ad vectors, that the expression of goal genes is adjustable by RU486. RU486 is added only when the anti-cancer gene expression is required. Namely if not necessary the anti-cancer gene expression can be closed. These GL-Ad vectors have no immunogenicity and can be existed in vivo for a long time;

4. This invention provides method for the construction and package of one kind of GL-Ad vectors. This method may use in construction and package of other GL-Ad vectors, and is performed easily;

5. This invention provides one kind of GL-Ad vectors, that the anti-cancer genes are easily inserted into. Many kinds of GL-Ad vectors expressing different anti-cancer genes can be constructed with this kind of GL-Ad vectors. This provides the good foundation for gene therapy of tumor;

6. In this invention, many kinds of GL-Ad vectors expressing different anti-cancer genes are constructed and the cell experiments certificate that these GL-Ad vectors are able to selectively kill the cancer cells but no affect on the normal cells. These noval GL-Ad vectors build the good foundation for the therapy of tumor from now on.

### BRIEF DESCRIPTION OF THE FIGURES

FIG. 1. The schematic drawing of construction and principle of the novel GL-Ad carrying two expression cassettes of this invention. (1) Schematic presentation of the organization of the Ad viral genome. (2) GL-Ad vector with the cis-acting ITRs and the packaging signal. (3) The cancer-targeting and inducible gene-expression GL-Ad contains two cassettes that 3.1 is the trans-activator(TA) cassette (cassette-1) and 3.2 is the anti-cancer gene expression cassette (cassette-2), the cis-acting ITRs and the packaging signal. (4) The action mechanism of the two expression cassettes.

FIG. 2. The Cre-loxP system of the helper virus for producing GL-Ad. The helper virus is bearing a packaging signal (Ψ) flanked by loxP sites. Cre-mediated excision of Ψ renders the helper virus genome unpackageable, but still able to replicate and provide all of the necessary trans-acting factors for propagation of the GL-Ad.

FIG. 3. An overview of examination on the targeting and adjustable expression of genes inserted in GL-Ad.

### DETAILED DESCRIPTION OF THE INVENTION

### Example I

Construction of the Shuttle Plasmid pRS-hTERT/Trail

1. Materials and Methods:

1-1. pRS-17: a plasmid containing the transactivator (TA), a chimeric regulator consisting of a mutated human progesterone receptor ligand binding domain (LBD) fused to the yeast GAL4 DNA binding domain and transcriptional activation domain of the human NF-kB p65 protein, 4×17-mer GAL4 upstream activation sequence, TATA cassette and a multiple cloning site into where genes of interested can be cloned.

Construction of pRS-17

(a) The larger HindIII/XbaI fragment was cut out from pGL3-Basic (Promega, USA) and then bluntened by Klenow enzyme and self-ligased. So the gene coding luciferase was deleted and the resulting plasmid was named pGL3-BasicΔ Luc.

(b) The unique ClaI site in the plasmid pSwitch (Invitrogen) was removed by site-directed mutation. The Stratagene QuikChange® II Site-Directed Mutagenesis Kits was used according to the manual. The complemental pair of oligonucleitide used were:

(1) 5> ccgagattgatgtcgacccc <3;
(2) 5> ggggtcgacatcaatctcgg <3.

(c) Gal4UAS fragment was amplified from the mutated pSwitch with the followed primer:

(1) 5> atttgcggccgccggagtactgtcctccgag <3
(2) 5> cgacgcgtttcgaggccacacgcgtc <3

(cycling condition: 94° C. 1 min, 56° C. lmin, 72° C. 30 sec, 30 cycles; Pfu Turbo polymerases from Stratagene were used)

The amplified fragment was digested by NotI/MluI and inserted into the corresponding site of the vector pUC6S (GeneBank ID: M74308) and the resulting plasmid was named pUC6S/Gal4. The MluI/ClaI fragment of pGL3-BasicΔ Luc containing the SV40 poly(A) sequence was cloned into the pUC6S/Gal4 to generate pUC6S/Gal4-poly(A).

(d) Transactivator(TA)-BGH poly(A) was amplified from pSwitch using the followed primers:

(1) 5> ccatcgatggtaccatggactcccagcagccagatc <3;
(2) 5> aaaggcgccgcggccgccagctggttctttccgc <3

(Cycling condition: 94° C. 1 min, 55° C. 1 min, 72° C. 3 min, 30 cycles)

This PCR fragment was digested by NarI/ClaI and insered into the ClaI site of pUC6S/Gal4-poly(A). Because NarI and ClaI have compatible cohesive ends, it was needed to screen the clones with the right orientation for retention of the ClaI site to be located between the two expression cassettes, and this correct plasmid was named pRS-17 (the progesterone regulatable system clone 17). The pRS-17 has many site of restriction endonucleases, therefore it is commonly used with single-restriction endonuclease digested cloning, bluntened connection and the gene transformation in different vectors on the vector construction.

1-2. The Plasmid pShuttle/hTERT: Construction Procedure of a Plasmid Carrying the hTERT Promoter

(a) pShuttle-CMV (Stratagene) was modefied by hTERT promoter replacing its naive CMV promoter. Firstly, the fragment amplified from pShuttle-CMV using the followed primers:

(1) 5> gcatggatccatatgcggtgtg <3
(2) 5> cggggtacctgagtaacacaaaattattc <3

(Cycling condition: 94° C. 1 min, 55° C. 1 min, 72° C. 1 min, 30 cycles) was digested by BamHI/KpnI and replaced the BamHI/KpnI fragment of pShuttle-CMV containing the CMV promoter, resulting of pShuttle.

(b) According to the reference of Cancer Research (59:551-557, 1999), the core element of hTERT promoter (−378-+78) was amplified using human gemome DNA library as template with the followed primers:

(1) 5> taggtaccctcccagtggattcg <3,
(2) 5> taggtacccagggcttcccacgtg <3

(Cycling condition: 94° C. 1 min, 58° C. 1 min, 72° C. 1 min, 30 cycles).

The fragment was digested by KpnI and inserted into the unique KpnI site of pShuttle and screened the clones with the right orientation.

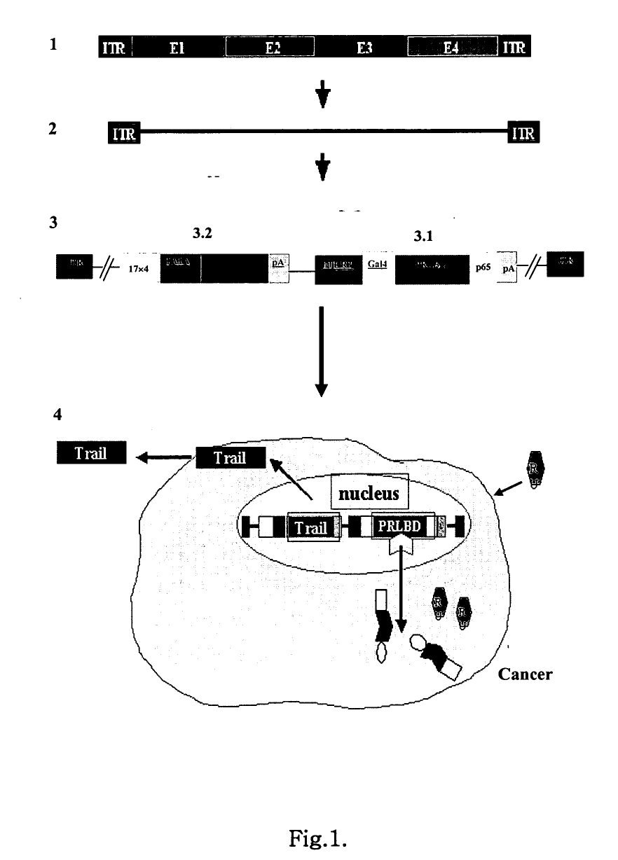

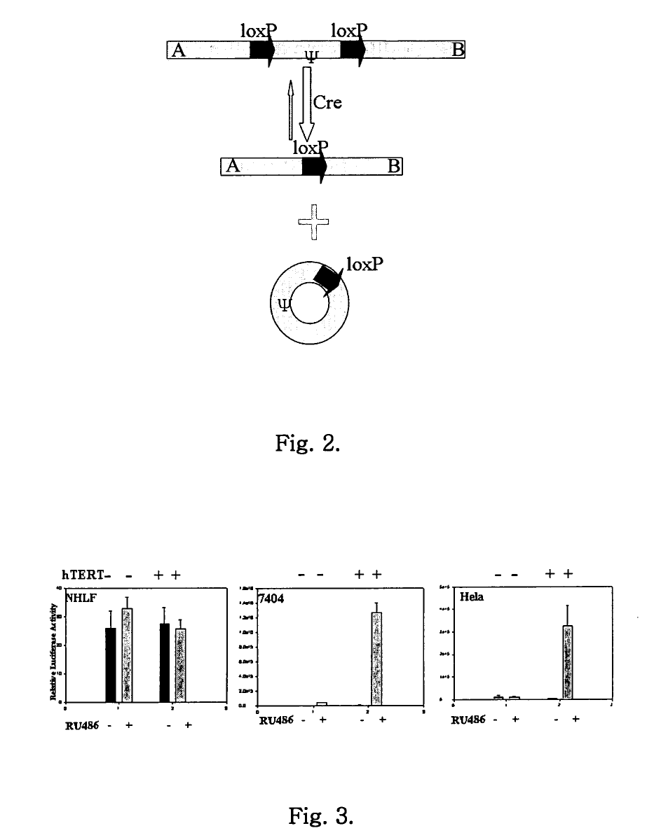

A schematic overview of construction procedure for the present noval GL-Ad with two regulated cassettes is shown in FIG. 1.

2. Construction Procedure

2-1. Construction of Cassette-1

The hTERT was cut from pShuttle/hTERT and cloned into the unique KpnI site of pRS-17 to generate pRS-hTERT, orientatining the hTERT to regulate expression of TA.

2-2. Construction of Cassette-2

TRAIL was cut from pCA13-Trail with HindIII/XbaI and cloned into a shuttle plasmid pGL3-Basic at HindIII/XbaI site to form plasmid pGL3-TRAIL. pGL3-TRAIL was cut with NheI/ClaI and digested fragment was inserted into the corresponding site of pRS-hTERT to make pRS-hTERT/Trail.

In the methods described above, hTERT promoter can be replaced by:
1) The telomerase reverse transcriptase, the catalytic subunit of the telomerase
2) α-fetoprotein (AFP) promoter
3) The carcinoembryonic antigen (CEA) promoter
4) Prostate-specific antigen (PSA) promoter
5) Breast-specific antigen (DF3/MUC-1) promoter

Detailed cloning technique is not described here because it is easy to operate by person of this field.

Moreover, in the regulated cassette-2, TRAIL can be replaced by the following genes with anti-tumor effect.

(1) TNF family: One member of this family, TRAIL(TNF-related apoptosis-inducing ligand) can bind to its receptor on the cell membrane and then cause apoptosis specifically in cancer cells.

(2) Tumor suppressor gene: Tumor supressor genes, including p53, Rb, NF1(Nuclear factor 1), VHL(von Hippel-Lindau), APC(Adenomatous polyposis coli), can repress the proliferationg of cancer cells.

(3) Cytokines with anti-tumor effect: Cytokine: Cytokines have many roles in regulating cell growth and death. Many cytokines can specifically kill tumor cells, activate immune cells and increase hematopoiesis, such as IL-2. IL-12, IL-24, GM-CSF, INF-α, IFN-β, IFN-γ.

(4) Pro-apoptotic genes: Cellular apoptosis plays very important roles in many life activities, and Many of today's medical illnesses can be attributed directly or indirectly to problems with apoptosis. And researchers have founded that most tumors have lower apoptosis or completely defective apoptosis machinery. Pro-apoptotic genes can accelerate the apoptosis of tumors and represent a new therapeutic protocol, such as Bax, Caspase family and Smac.

(5) Antiangiogenesis-related genes: The antiangiogenesic gene suppress tumor newborn vascularization to block the nutrition supply for tumor cells. The tumor will wither and die because of the malnutrition. The antiangiogenesic gene includes angiostatin and endostatin.

(6) Suicide genes: herpes simplex virus-thymidine kinase, bacterial cytosine deaminase, purine nucleotide phosphorylase (PNP), thymidine phosphorylase (TP), deoxycytidine kinase (dCK).

(7) Other genes that have antitumor effects: Others: Sflt-1, soluble variant of the Vascular Endothelial Growth Factor Receptors Flt-1, can competently supress the function of VEGF.

The goal gene is flanked by ClaI sites by mediate clones (in order to insert into ClaI site behind the expression cassette-2), or the front end of the goal gene is ClaI site and the opposite end is a bluntened end (in order to insert into ClaI/SwaI sites behind the expression cassette-2), or the both ends of the goal gene are bluntened by the Klenow polymerase in order to insert into SwaI site. Detailed cloning technique is not described here because it is easy to operate by person of this field.

### Example II

Construction of the Packaging Plasmid pGL-hTERT/Trail

1. Materials and Principles

The packaging plasmid pGL (Originally constructed by Merk Research Laboratories, and can be obtained from Microbix, Canada) harbors the cis-elements needed for the replication and package, the LITR, the RITR and the packaging signal (Ψ) of type 5 Ad5. Parts of the HPRT and human cosmid 346 were incorporated as the stuffer sequence. An unusual restriction enzyme, PmeI, can be linearized the packaging plasmid pGL to expose ITR sequences at both ends, and at the same time also removed resistant screening marker and duplication starting point sequence belonging to prokaryotic cell in the pGL. In the pGL, there is an unusual restriction enzyme, EagI, which is convenient for the insertion of foreign DNA.

2. Experimental Procedure

The NotI fragment containing the two cassettes of pRS-hTERT/Trail was cloned into the unique EagI site (NotI and EagI have compatible cohesive ends) of pGL to make the final packaging plasmid pGL-hTERT/Trail.

### Example III

Package of Gutless Adenoviral Vector

1. Experimental Material and Principle

Helper virus H14 and Packaging cell 293Cre4 were obtain from Microbix Biosystem Inc., Toronto. Helper virus H14 was a modefied first generation adenoviral vector. The packaging signal was flanked by two loxP sequence. When infecting the packaging cell 293Cre4, homologous recombination took place. The packaging signal of the helper virus was lost and cannot package in the 293Cre4. The packaging cell 293Cre4 can stably expresses Cre recombinase. When linearized pGL-hTERT/Trail was cotransfected/infected with the helper virus H14. The helper virus can provide the gutless virus with all the proteins for replication and package, but the helper virus itself cannot package into viron paticles because the packaging signal was removed in the presence of the Cre recombinase. So pGL-hTERT/Trail contains the packaging signal and can package into GL-Ad using the proteins for replication and package provided by the helper virus when linearized pGL-hTERT/Trail was cotransfected/infected with the helper virus.

2. Experimental Procedure:

2-1. Amplification and Purification of GL-Ad Vector

(1) Amplification #1: pGL-hTERT/Trail is completely digest with PmeI, extracted with phenol/trichloromethane and the DNA is precipitated with the ethyl alcohol. This DNA was transfected into 293Cre4 cells. After 6 h post-transfection, remove the medium from the transfected monolayer and immediately infect the transfected cells with the helper virus at an MOI of 5 PFU/cell. When the complete cytopathic effect (CPE) was observed by about 48 h postinfection, the cells are harvested, subject the cell suspension to three rounds of freeze/thaw and stored at −80° C.

(2) Amplification #2: Infect Cre-expressing cells on one 60 mm dish with ½ of the lysate from amplification #1, add 5 MOI of helpervirus. When the complete cytopathic effect (CPE) was observed by about 48 h postinfection, the cells are harvested, subject the cell suspension to three rounds of freeze/thaw. Infect Cre-expressing cells again with this suspension, and then add 5 MOI of helpervirus. Repeat this procedure 4-5 times.

(3) Amplification #3: Cells harvested from the final time of amplification #2 are spun down for 20 min at 1500 rpm, then dissolved in 2 ml Tris Saline. The cell suspension to three rounds of freeze/thaw is down and keep supernatant at −80° C.

(4) DNA is extracted from the supernatant (200 μl) obtained in amplification #3 using QIAamp DNA Mini Kit (Qiagen) according ot the manufacturer. Eluted DNA should be precipitated with ethanol and dissolved in 10 μl TE. Use 5 μl of the vector DNA and 200 ng of the corresponding GL-Adv plasmid, digest with an appropriate enzyme and run on 0.8% agarose gel containing EtBr. If the bands corresponding to vector DNA are clearly visible and are of at least “equal” intensities to fragments corresponding to helpervirus DNA, a large scale preparation can be performed. If no vector DNA can be detected, restarting from amplification #1.

2-2. Large Scale Preparation and Purification of Gutless Adenoviral Vector

Infect more Cre-expressing cells with the lysate from amplification #3, and then add 5 MOI of helper virus. When the complete cytopathic effect (CPE) was observed by about 48 h postinfection, the cells are harvested, subject the cell suspension to three rounds of freeze/thaw. GL-Ad is seperated by CsCl gradient centrifugation.

The pollution rate of helper virus may be estimated by the relative quantity of plaque forming (plaque shapes formed by helper virus and GL-Ad are different), or can compare the proportion of the two with Southern Blot. In our system, the Cre-loxP recombinant system is used. The Cre recombinase which is expressibg in the packing cell can effectively excise the packaging signal from helper virus, thus suppress its packaging. This may control the contamination of helper virus in 0.2%.

### Example IV

Examination of Targeting and Adjustable Expression of Genes Inserted in GL-Ad

Construct the GL-Ad carrying luciferase reporter gene according as above. Particular steps are as follows: The luciferase gene sequence was released with NheI and ClaI from pGL3-Enhancer(Promega) and ligated into pRS-hTERT. The fragment containing luciferase sequence of pRS-hTERT/Luc cutted by NotI was inserted into SwaI site of pGL and generated pGL-hTERT/Luciferase. After packaged in 293 Cre cell, the GL-Ad-hTERT/Luciferase infected normal and cancer cell lines respectively, and was induced by RU486 (mifepristone) subsequently. The quantificational measure of Luciferase gene expression was accomplished by refering to the product information of Promega. The results were showed in FIG. 3.

According to the results in FIG. 3, the activity of luciferase had no obvious difference in normal cell NHLF whether induced by RU486 and controlled by hTERT promoter or not, and both of the activity levels were low. However, in cancer cells (7404 and Hela), the activity of luciferase were highest under the condition of induced by RU486 and controlled by hTERT promoter. Therefore, the GL-Ad constructed by the present invention has two traits: cancer-targeting and inducible.

In the cancer-targeting and inducible GL-Ad from the present invention, the trans-activator(TA) cassette controlled by tumor-specificity promoter consists of a mutated human progesterone receptor ligand binding domain(LBD) fused to the yeast GAL4 DNA binding domain and transcriptional activation domain of the human NF-kB p65 protein, and the ending signal. The expression product of TA is a chimeric protein , of which GAL4 DNA binding domain can bind 17mer×4 sequence. The mutant LBD of TA can not bind normal endogenetic ligand, but RU486. After combining RU486, the changing conformation of LBD makes the chimeric protein possess of the ability of binding 17mer×4 sequence, and ultimately activates the transcription of interesting gene. Without combining with RU486, the chimeric protein has no the transcriptional activation function. Thus, the activity of TA is regulated by two factor: regulated by tumor-specificity promoter in transcriptional and translational level, and by small molecule drug RU486 in functional exertion.

GAL4 is a transcriptional factor from yeast and far away from human, which can not identify the human transcriptional regulating domain , therefore, it is prevented TA from interference with human gene.

The cassette-1 expression is controlled by the promoter hTERT. It's product (TA element )is inactive, only after changing the conformation by combined with RU486. The activated TA binds 17mer×4 of anti-cancer cassette upsteam sequence, and activates the expression of the anti-cancer gene. So the anti-cancer gene expression is dual controlled. First, it is controlled by hTERT, TA is targetingly expressed in cancer cell but not in normal cell. Second, it is regulated by RU486. TA is inactive without RU486. Thus, adds RU486 when only the anti-cancer gene expression is required, namely if not necessary the anti-cancer gene expression can be closed to reduce side-effect greatly.

What is claimed:

1. A gutless adenovirus vector (GL-Ad vector), which is cancer-targeting and adjustable expression of foreign genes, is constructed by inserted the two regulated cassettes into GL-Ad vector, One is the trans-activator(TA) cassette controlled by tumor-specific promoter, the other is the anti-cancer gene expression cassette controlled by TA and RU486.

2. The GL-Ad vector of claim 1, wherein the trans-activator(TA) cassette includes: (1) tumor-specific promoter; (2) the trans-activator(TA) consists of a mutated human progesterone receptor ligand binding domain(LBD) fused to the yeast GAL4 DNA binding domain and transcriptional activation domain of the human NF-kB p65 protein, and the ending signal.

3. The GL-Ad vector of claim 1, wherein the anti-cancer gene expression cassette includes: (1) the yeast GAL4 upstream 17mer×4 sequence; (2) TATA cassette; (3) anti-cancer gene downstreasm the TATA cassette; (4) and the ending signal.

4. A construction method of the GL-Ad vector of any one of claims 1-3, wherein the method includes the following steps:
(1) Construction of the plasmid pRS-hTERT/Trail
(1)-1. Construction of the trans-activator(TA) cassette (cassette-1)
The hTERT was cut from pShuttle/hTERT and cloned into the unique KpnI site of pRS-17 to generate pRS-hTERT, orientatining the hTERT to regulate expression of TA.
(1)-2. Construction of the anti-cancer gene expression cassette (cassette-2)
TRAIL was cut from pCA13-Trail with HindIII/XbaI and cloned into a shuttle plasmid pGL3-Basic at HindIII/XbaI site to form plasmid pGL3-TRAIL. pGL3-TRAIL was cut with NheI/ClaI and digested fragment was inserted into the corresponding site of pRS-hTERT to make pRS-hTERT/Trail.
(2). Construction of the packaging plasmid pGL-hTERT/Trail
The two cassettes were cut out with NotI from plasmid pRS-hTERT/Trail and cloned into the unique EagI site of pGL to make the final packaging plasmid pGL-hTERT/Trail.
(3). Package of gutless adenoviral vector
Linearized pGL-hTERT/Trail was cotransfected/infected the 293Cre4 cell with the helper virus H14 (Microbix Biosystem Inc., Toronto) to package the novel GL-Ad with cancer-targeting and adjustable expression of genes.
(4). Large scale preparation and purification of GL-Ad
After several rounds of plaque purification and the cloning is correct, this GL-Ad stock is used for large scale preparation. 293Cre4 cells are infected with this GL-Ad stock. When cells show complete CPE, cells are harvested. Purified GL-Ad vector is obtained from CsCl gradient centrifugation and freeze in aliquots at −80° C.

5. The construction method of the GL-Ad vector of claim 4, wherein hTERT promoter in regulator cassette-1 can be replaced by:
1) The telomerase reverse transcriptase, the catalytic subunit of the telomerase;
2) χ-fetoprotein (AFP) promoter;
3) The carcinoembryonic antigen (CEA) promoter;
4) Prostate-specific antigen (PSA) promoter;
5) Breast-specific antigen (DF3/MUC-1) promoter.

6. The construction method of the GL-Ad vector of claim 4, wherein in the regulated cassette-2, TRAIL can be replaced by the following genes with anti-tumor effect: (1) TNF family gene; (2) Tumor suppressor gene; (3) Cytokines with anti-tumor effect; (4) Pro-apoptotic gene; (5) Antiangiogenesis-related gene; (6) Suicide gene.

7. The construction method of the GL-Ad vector of claim 6, wherein tumor supressor genes include p53, Rb, NF1(Nuclear factor 1), VHL(von Hippel-Lindau), or APC(Adenomatous polyposis coli) which can repress the proliferationg of cancer cells.

8. The construction method of the GL-Ad vector of claim 6, wherein cytokines genes with anti-tumor effect include IL-2, IL-12, IL-24, GM-CSF, INF-α, IFN-β, IFN-γ.

9. The construction method of the GL-Ad vector of claim 6, wherein pro-apoptotic genes include Bax, Caspase family and Smac.

10. The construction method of the GL-Ad vector of claim 6, wherein antiangiogenesis-related genes include angiostatin and endostatin.

11. The construction method of the GL-Ad vector of claim 6, wherein suicide genes include herpes simplex virus-thymidine kinase, bacterial cytosine deaminase, purine nucleotide phosphorylase (PNP), thymidine phosphorylase (TP), deoxycytidine kinase (dCK).


SEQUENCE LISTING (10 sequences)

SEQ ID NO: 1 - DNA, 20 bp, Artificial
  Oligonucleotide for site-directed mutagenesis of ClaI site in pSwitch
ccgagattgatgtcgacccc

SEQ ID NO: 2 - DNA, 20 bp, Artificial
  Oligonucleotide for site-directed mutagenesis of ClaI site in pSwitch
ggggtcgacatcaatctcgg

SEQ ID NO: 3 - DNA, 31 bp, Artificial
  Oligonucleotide for amplification of Gal4UAS fragment from mutated pSwitch
atttgcggccgccggagtactgtcctccgag

SEQ ID NO: 4 - DNA, 26 bp, Artificial
  Oligonucleotide for amplification of Gal4UAS fragment from mutated pSwitch
cgacgcgtttcgaggccacacgcgtc

SEQ ID NO: 5 - DNA, 36 bp, Artificial
  Oligonucleotide for amplification of transactivator(TA)-BGH poly(A) from pSwitch
ccatcgatggtaccatggactcccagcagccagatc

SEQ ID NO: 6 - DNA, 34 bp, Artificial
  Oligonucleotide for amplification of transactivator(TA)-BGH poly(A) from pSwitch
aaaggcgccgcggccgccagctggttctttccgc

SEQ ID NO: 7 - DNA, 22 bp, Artificial
  Oligonucleotide for amplification of CMV promoter from pShuttle-CMV
gcatggatccatatgcggtgtg

SEQ ID NO: 8 - DNA, 29 bp, Artificial
  Oligonucleotide for amplification of CMV promoter from pShuttle-CMV
cggggtacctgagtaacacaaaattattc

SEQ ID NO: 9 - DNA, 23 bp, Artificial
  Oligonucleotide for amplification of hTERT promoter
taggtaccctcccagtggattcg

SEQ ID NO: 10 - DNA, 24 bp, Artificial
  Oligonucleotide for amplification of hTERT promoter
taggtacccagggcttcccacgtg